(19) United States
(12) Patent Application Publication
(10) Pub. No.: US 2026/0029366 A1
(43) Pub. Date: Jan. 29, 2026

(54) REGENERATION METHOD FOR AN ELECTROCHEMICAL SENSOR AND CORRESPONDING SENSOR ARRANGEMENT

(71) Applicant: Testo SE & Co. KGaA, Titisee-Neustadt (DE)

(72) Inventors: Gregor STEINER, Titisee-Neustadt (DE); Johannes DORNHOF, Kirchzarten (DE)

(73) Assignee: Testo SE & Co. KGaA, Titisee-Neustadt (DE)

(21) Appl. No.: 19/268,175

(22) Filed: Jul. 14, 2025

(30) Foreign Application Priority Data
Jul. 29, 2024 (EP) 24191528.9

(51) Int. Cl. G01N 27/38; G01N 27/28; G01N 27/30; G01N 33/00

(52) U.S. Cl. CPC G01N 27/38; G01N 27/283; G01N 27/301; G01N 33/0029; G01N 33/0037; G01N 33/004

(57) ABSTRACT

A regeneration method for an electrochemical sensor (1), in which the sensor (1) includes at least two electrodes (2), the at least two electrodes (2) are operated in a first connection in a normal operation of the sensor (1). The regeneration method differs from the normal operation in terms of a modified connection of the at least two electrodes (2).

### CROSS-REFERENCE TO RELATED APPLICATIONS

This application claims priority from European Patent Application No. 24191528.9, filed Jul. 29, 2024, which is incorporated herein by reference as if fully set forth.

### TECHNICAL FIELD

The invention relates to a regeneration method for an electrochemical sensor, wherein the sensor comprises at least two electrodes, wherein the at least two electrodes are operated in a first connection in a normal operation of the sensor.

The invention further relates to a sensor arrangement having at least one sensor, wherein the sensor comprises at least two electrodes.

### BACKGROUND

It has been found that such electrochemical sensors, in particular electrochemical gas sensors, occasionally become contaminated, in particular by organic vapors or organic agents used in cleaning operations. This contamination can interfere with the correct function of the gas sensor, which means that, for example, high gas concentrations can no longer be detected or are no longer correctly detected. Such a malfunction can, for example, have life-threatening consequences in the case of carbon monoxide sensors (CO sensors) or nitrogen monoxide sensors (NO sensors) and must always be avoided.

The normal recommendation for handling these types of substances is to keep them away from the sensor, but, in practice, contamination with such substances or other substances occurs.

Regeneration methods are known which comprise temperature control, in which case a long treatment of the sensor is required in order to remove contamination.

### SUMMARY

The invention is concerned with restoring the original operating state of the sensor as quickly as possible.

This object is achieved by a regeneration method having one or more of the features disclosed herein. To achieve said object, it is thus provided according to the invention in a regeneration method of the aforementioned type that the regeneration method differs from normal operation in terms of a modified connection of the at least two electrodes. In this way, substances which have formed and which change the response behavior of the sensor due to the potential shifts can be eliminated.

The modified connection can be configured, for example, to effect at least one substance conversion at the at least two electrodes which differs from normal operation. Thus, for example, aging and/or contamination processes can be reversed again at least partially or even almost completely (for example, with the exception of a deviation typical of production tolerance).

The regeneration method can be implemented, for example, by means of a potentiostat. Desired electrochemical voltage conditions can thus be easily set, in particular to effect a substance conversion.

It is particularly advantageous in this case that, for example, a shift of a working point of the sensor, as a result, for example, of contamination of an electrode, can be detected and rectified. Furthermore, it is particularly advantageous that the risk of measurement inaccuracies and/or measurement errors can thus be minimized and the original operating state can be quickly restored.

In a further advantageous embodiment, it can be provided that at least one first electrode is suitable for detecting contamination of a second electrode. Thus, on the basis of anomalous and/or abnormal signals at the auxiliary electrode, contamination of the sensor can be detected at an early stage and countermeasures can be initiated. For example, the signal of the auxiliary electrode of an NO sensor in the presence of organic vapors is up to two orders of magnitude higher than in the case of exposure to NO gas. In addition, in this example of an NO sensor, the sensing electrode has no direct response behavior in the presence of organic vapors, so that it cannot indicate contamination.

In a further advantageous embodiment, it can be provided that modifying the connection comprises applying a current to the first electrode, in particular to generate a substance conversion from the first electrode, wherein the substance conversion preferably does not take place at this electrode in normal operation.

The electrode to which current is applied can be, for example, a reference electrode, which is integrated, in particular high-ohmically, into the three-electrode structure in the normal operating mode of an electrochemical cell. Electrodes with an equilibrium potential that is both constant and can be set quickly and reproducibly are referred to as reference electrodes. Such reference electrodes are used, for example, as a reference point for the measurement of preferably relative potentials of further electrodes.

It is particularly advantageous that substances formed at the reference electrode, which, in particular due to a potential shift, for example, change a reaction behavior of the sensor, can be detected and corresponding countermeasures can be initiated.

In a further advantageous embodiment, it can be provided that the potential level is determined in an upstream self-test. The operating point of the sensor can thus be determined and the true or actual potential level of the reference electrode can be determined via the potential value of the reduction peak.

In one advantageous embodiment, it can be provided that a current flow is determined as a function of the applied voltage, in particular in a linear sweep method. The operating point of the sensor can thus be determined and the actual potential level of the reference electrode can be determined on the basis of the peak.

Such a self-test can be carried out, for example, by means of a linear sweep to determine the current flow as a function of the applied voltage. The linear sweep is a (preferably monotonic, for example monotonically increasing or monotonically decreasing) variation in the applied voltage, wherein the counter electrode is operated as a sensing electrode and the auxiliary electrode as a counter electrode in the electrode connection. The self-test can be used to determine a peak value. Depending on the type of sensor and the materials from which the electrodes are made, it is possible to use the sensing electrode as a counter electrode instead of the auxiliary electrode. The attainment of a peak value, for example, is usable as a condition to switch, preferably automatically, to the next connection and/or to output diagnostic values. A diagnostic value here can be the confirmation of an unimpaired operating point, i.e. a completely intact sensor.

In an NO sensor, for example, the sensing electrode is made of carbon if a selective detection of nitrogen monoxide (NO) without carbon monoxide (CO) is desired. The auxiliary electrode and counter electrode can be made of platinum here.

For a carbon monoxide (CO) measurement, the sensing electrode is also made of platinum, so that the sensing electrode can be used in this case as a counter electrode.

However, the specified materials are highly sensor-specific and depend on the field of application, and are to be understood here merely as examples.

The determined peak value can be indicative of the voltage at which the optimal substance conversion takes place. At voltages above the peak value, for example, it is not possible to generate a sufficient voltage for a substance conversion and, at voltages below the peak value, the sensor is no longer able to convert additionally offered substances in a short time.

It is particularly advantageous that the peak value can be used to characterize the sensor. The sensor is no longer able, for example, to convert additionally offered substances in a short time if the determined peak value is too close to the normal operating point of the sensor. If the concentration of the detected substance or converted substance changes, for example, the sensor reacts only very slowly because it is busy breaking down the material offered to it. If the determined peak value is very far away from the normal operating point of the sensor, the sensor has sufficient capacity to quickly break down existing substances and can therefore react very quickly to changes in concentration.

In normal operation, e.g. In the case of an oxygen sensor, the working value is selected at −600 mV. In order to avoid cross-sensitivities, such as nitrous oxide, the working value can also be selected at a value of −300 mV.

It is particularly advantageous that the shift of the peak value indicates that an undesired substance conversion and/or substance input has taken place at other electrodes, which can lead to a change in the potential levels.

Furthermore, the chemical composition of the sensor may have changed, so that, for example, electrochemical voltages which should not be there occur between certain electrodes.

In a further advantageous embodiment, it can be provided that a current flow is determined as a function of an applied voltage between two of the at least three electrodes of the sensor, preferably between a second and a third electrode of at least three electrodes of the sensor, particularly preferably between a counter electrode and auxiliary electrode or between a sensing electrode and the auxiliary electrode of the sensor. The counter electrode is used as a sensing electrode or measuring electrode and the auxiliary electrode is used as a counter electrode.

In a further advantageous embodiment, it can be provided that a relative potential difference between two of the at least three electrodes of the sensor is measured. The potential difference can be determined, for example, by an open connection potential (OCP) measurement. The potential value between the reference electrode and the counter electrode can thus be determined by means of a potentiometric measurement. During the OCP measurement, no electrical current flows, as a result of which no voltage drop occurs via the internal resistance of the voltage source. The OCP measurement is a passive measurement method, which means that the counter electrode (which is necessary to pass current through the cell) in the potentiostat connection is bypassed. In this mode, only the rest potential between the reference and sensing electrodes is measured. However, in the potential measurement described here as an example, the counter electrode acts as a sensing electrode.

In a further advantageous embodiment, it can be provided that a relative potential difference between the first electrode and a second or third electrode of at least three electrodes of the sensor, particularly preferably between a reference electrode and a counter electrode of the sensor is measured. This enables detection of electrochemical potential differences which should not occur in the normal operation of the sensor. Attainment of a stationary potential difference can be used, for example, as a condition for a, preferably automatic, changeover to a next connection and/or to an output of at least one diagnostic value.

In a further advantageous embodiment, it can be provided that at least one electrode is amperometrically energized. It is thus possible to determine the duration of the energization and the level of energization required to regenerate the sensor and thus restore its functionality. The energization of the reference electrode has been shown to be particularly advantageous for restoring functionality. This can be done, for example, at least in the modified connection.

Especially in the case of the oxygen sensor (O2 sensor), it has been shown that the energization of the reference electrode is the preferred method for direct and complete regeneration of the sensor. Due to contamination, the O2 sensor can enter an incorrect measurement mode in which the potential level at the reference electrode is shifted (or lowered) to the extent that oxygen is no longer reduced at the sensing electrode, but instead hydrogen is generated through water electrolysis. The hydrogen produced at the measuring electrode diffuses through the sensor body and causes the potential at the reference electrode to drop even more sharply so that the voltage measured between the reference electrode and counter electrode is increased by about 1 V, i.e. to around 1.5 V.

This faulty operation can be detected unequivocally by two measured variables. On the one hand, the measuring signal increases by about two orders of magnitude, and, on the other hand, the potential difference between the reference electrode and the counter electrode increases by around 1 V. The potential difference between the reference and counter electrode is thus around 1.5 V instead of only 0.5 V.

For the regeneration of the sensor, the potential difference between the reference electrode and counter electrode is used as the output variable for the anodic energization of the reference electrode. If the connection is modified after a potential voltage of 1.5 V is measured between the reference electrode and the counter electrode, and the counter electrode is connected as a sensing electrode, the reference electrode as a sensing electrode and the sensing electrode as a counter electrode, the potential level of the reference electrode is increased by approx. 900 mV at a selected potential voltage of −600 mV. Following a connection of this type, the sensor is operated for a period of approx. 10 minutes. After this energization interval, the oxygen sensor returns to its original operating state.

In a further embodiment, attainment of a determined current flow, which can indicate, for example, a complete substance conversion, can be used as a condition for a, preferably automatic, changeover to a next connection and/or to an output of at least one diagnostic value.

In a further advantageous embodiment, it can be provided that the amount of the applied current is determined based on the determined current flow, in particular the determined voltage value for the extreme value of the current flow, and/or the determined relative potential difference, preferably based on an addition of the voltage value and the potential difference. It is particularly advantageous if the reference electrode is energized as a sensing electrode, the counter electrode as a reference electrode and the auxiliary electrode as a counter electrode.

In a further advantageous embodiment, it can be provided that the reference electrode is not energized as described above through a targeted application of a potential, but through operation as a counter electrode. It is particularly advantageous if the reference electrode is operated as a counter electrode, the counter electrode as a reference electrode and the sensing electrode is operated unchanged. The charge and therefore the potential of the reference electrode, which is operated as a counter electrode, can then be shifted in the direction of the initial state by means of the current flowing through the electrochemical cell.

It is particularly advantageous if the reference electrode is energized during the energization variation. The applied potential can be calculated from the results of the actual potential level of the reference electrode and the OCP value, i.e. the measurement of the potential voltage between the counter electrode and the reference electrode.

In a further advantageous embodiment, it can be provided that a self-test is carried out to monitor the regeneration method. This makes it possible to monitor the effectiveness of the regeneration measure. It is particularly advantageous if such a regeneration method can be carried out automatically on the sensor, in particular if measured values which lie outside an expected range are generated in normal operation.

In a further advantageous embodiment, it can be provided that a first electrode is a sensing electrode, a second electrode is an auxiliary electrode, a third electrode is a counter electrode and/or a fourth electrode is a reference electrode of the sensor. It is thus possible, by means of a modified connection of the electrodes, to initiate a regeneration method which differs from the connection of the electrodes in the normal operating mode. This can be done, for example, by applying a current to an electrode that is used for voltage measurement in normal operation, for example in order to generate a substance conversion that does not usually take place at this electrode in normal operation.

In a further advantageous embodiment, it can be provided that the sensor is a nitrogen monoxide sensor (NO sensor) or a carbon monoxide sensor (CO sensor).

This object is alternatively or additionally achieved by a sensor arrangement having one or more of the features disclosed herein, which focus on a sensor arrangement. In order to achieve said object, it is provided according to the invention, in a sensor arrangement of the aforementioned type, that a connection device is provided with which at least two connections of the at least two electrodes can be changed. Different processes in the electrochemical sensor are thus externally activatable. No mechanical, chemical and/or thermal cleaning, which may require the use of chemical substances, is therefore required.

The connections can be characterized, for example, by different assignments of the electrodes with potentials and/or with ground and/or by different voltage and/or current measurements at the at least two electrodes and/or by injecting a current into one connection and/or applying a voltage to another connection. This allows the electrodes to be easily switched externally to states or modes of operation that differ from the normal operation. This means that different, in particular reversed, substance conversions can be effected compared with a normal operation, for example the aforementioned normal operation.

It can be provided that a first connection of the at least two connections is configured for normal operation and/or a modified connection is configured for a regeneration method, in particular as described above and/or claimed below.

In one advantageous embodiment, it can be provided that the connection device is configured for an automatic, preferably condition-controlled, changeover between the at least two connections, in particular between at least three or at least four connections. More complex regeneration methods requiring a sequence of different connections are thus reproducible.

For example, the at least two, in particular at least three and/or at least four, connections from the group of:
normal operation (measurement and/or detection operation)
determination of a potential level of an electrode
performance of a linear sweep
OCP measurement
amperometric regeneration operation
optionally: checking the effectiveness of the performed regeneration
can be selected. This enables the implementation of one or more of the aspects described below.

The sensor arrangement according to the invention can thus be advantageously used to carry out a regeneration method according to the invention.

### BRIEF DESCRIPTION OF THE DRAWINGS

The invention will now be described in detail with reference to an exemplary embodiment, but is not limited to the exemplary embodiment. Further exemplary embodiments can be derived by combining the features of individual claims or a plurality of claims with one another and/or with individual features or a plurality of features of the exemplary embodiment.

In the figures:

FIG. 1 shows an exploded view of a sensor according to the invention,

FIG. 2 a schematic view of the mode of operation of an electrochemical sensor,

FIG. 3 shows a wiring diagram of the electrochemical sensor in normal operation,

FIG. 4 shows a connection diagram for performing a linear sweep to determine the potential level of the reference electrode of the electrochemical sensor,

FIG. 5 shows a connection diagram for OCP measurement to determine the potential difference between the counter electrode and the reference electrode of the electrochemical sensor, and

FIG. 6 shows a connection diagram for the amperometric regeneration energization of the electrochemical sensor.

### DETAILED DESCRIPTION

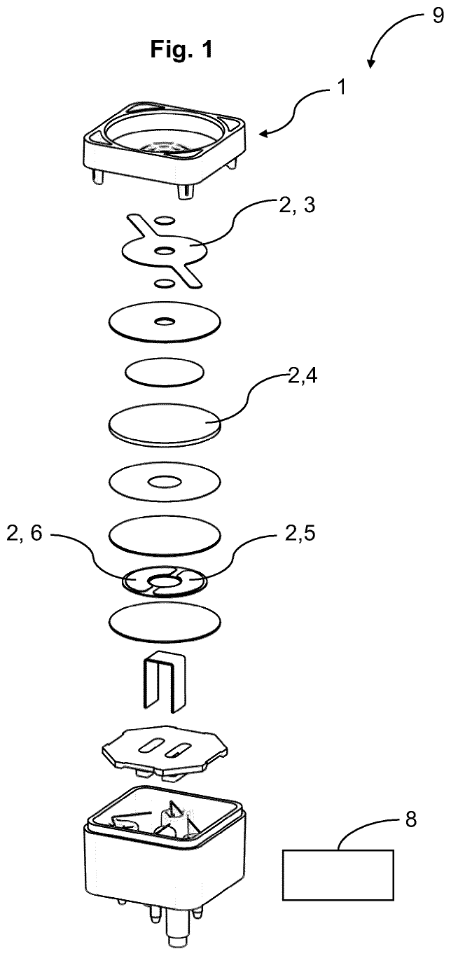

FIG. 1 shows an exploded view of a sensor 1, in particular a gas sensor. Such sensors 1 can be designed, for example, as nitrogen monoxide sensors (NO sensors) or carbon monoxide sensors (CO sensors).

In the example shown, the electrochemical sensor 1 has the following electrodes from top to bottom: a first electrode 2 (sensing electrode), a second electrode 3 (auxiliary electrode), a third electrode 4 (counter electrode) and a fourth electrode 5 (reference electrode). The intermediate layers are in each case separators or shielding membranes, which are not relevant to the method according to the invention and are therefore not further described.

The sensor 1 is part of a sensor arrangement 9, which further comprises a connection device 8.

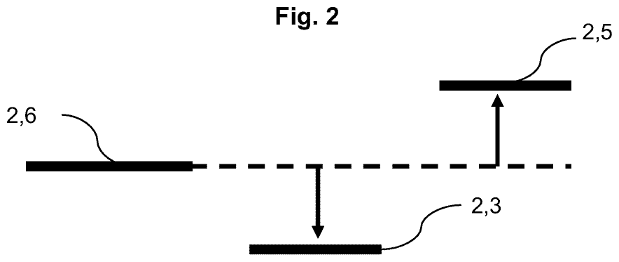

FIG. 2 shows a schematic view of the mode of operation of an electrochemical sensor. After energization, oxygen is reduced at the sensing electrode 3. The potential of the sensing electrode 2 is reduced by 600 mV compared with the reference electrode 6. As soon as the sensor starts to operate due to this energization, oxygen is reduced by lowering the potential at the sensing electrode 2. At the same time, a counter-reaction must take place to ensure charge balancing, which means that a corresponding counter-reaction takes place at the counter electrode 5. Water is electrolytically/anodically decomposed and oxygen is released. As a result, the potential of the counter electrode 5 increases by several hundred mV compared with the reference electrode 6. This potential distribution is stable in normal operation and with constant energization.

Contamination of the sensor 1 by organic substances can result in contamination of the electrochemical sensor 1 and can consequently lead to an incorrect measurement of the electrochemical sensor 1.

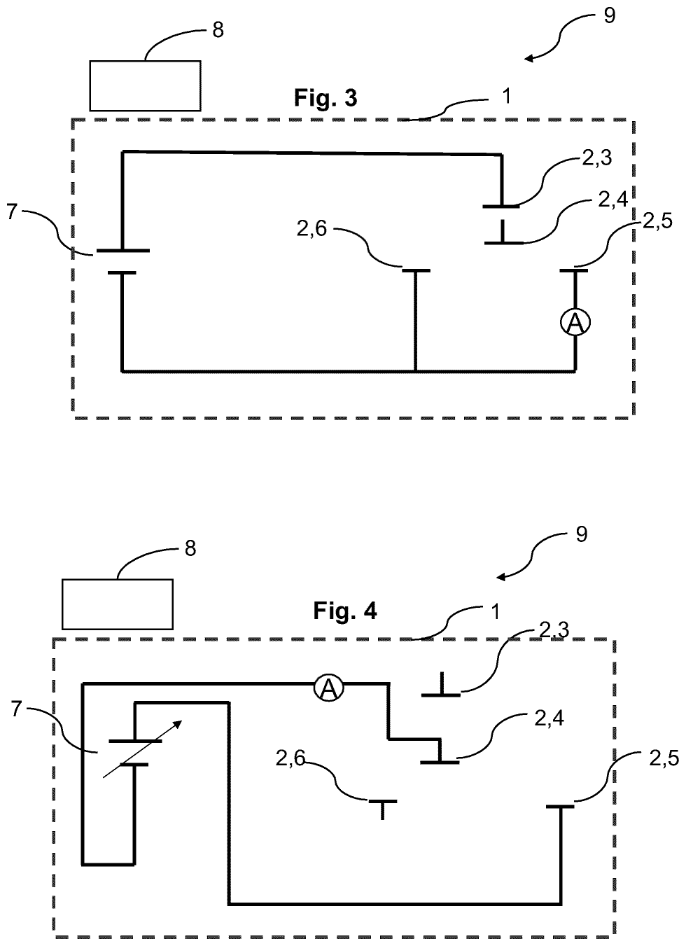

FIG. 3 shows a connection diagram of the electrochemical sensor 1 in normal operation with a voltage source 7. As already explained with reference to FIG. 2, oxygen is reduced at the sensing electrode 2 following energization. A counter-reaction must take place simultaneously for charge balancing. An opposite reaction takes place here at the counter electrode 5, i.e. water is electrolytically decomposed at the counter electrode 5 and oxygen is released.

The steps shown in FIG. 4 to FIG. 6 show an example of a regeneration method for restoring the original operating state of an electrochemical sensor 1.

A connection of the electrodes that differs from normal operation enables the performance of an accelerated restoration of the measuring capability following contamination of an electrochemical gas sensor 1.

The procedure is illustrated using the following steps of the exemplary embodiment for detecting and regenerating the gas sensor 1:

In the first step (not shown), the auxiliary electrode 3 can be used to detect contamination of the electrochemical sensor 1. To do this, the connection is modified compared with normal operation.

In this case, anomalous signals at the auxiliary electrode 4 can indicate deviations from the normal operating state of the electrochemical sensor 1. For example, the signal at the auxiliary electrode 4 in the presence of organic vapors can be up to more than two orders of magnitude higher than in normal operation, whereas the sensing electrode 3 shows no direct response behavior in the presence of organic vapors.

The measured variables/characteristics and/or a value derived therefrom can be output as a diagnostic value.

In the next step of the regeneration method, the energization quantity is determined in a self-test of the sensor 1. To do this, the connection is modified.

FIG. 4 shows the circuit in which the current flow is determined by means of the linear sweep method as a function of the applied voltage. The operating point of the sensor 1 is determined here. The counter electrode 5 is energized here as a sensing electrode 3 and the auxiliary electrode 4 as a counter electrode 5, so that the actual potential level of the reference electrode 6 can be determined via the potential value of the reduction peak.

The measured quantity and/or a value derived therefrom can be output as a diagnostic value.

In the further course of the regeneration method, a relative potential difference between two of the electrodes 2 is measured. To do this, the connection of the electrodes 2 is modified again.

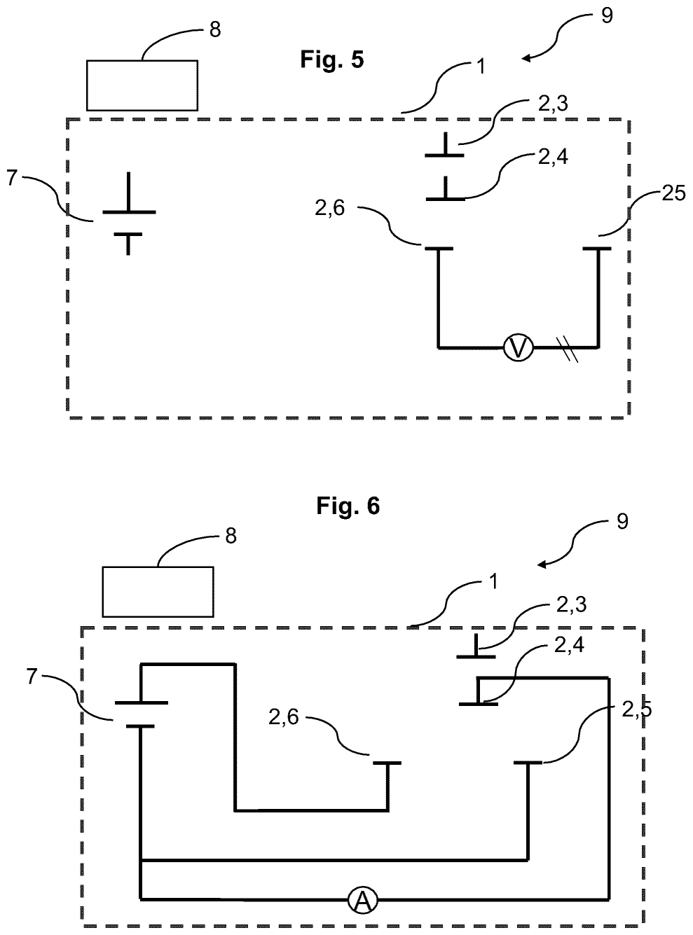

FIG. 5 of the exemplary embodiment according to the invention shows an open circuit potential (OCP) measurement. A measurement without current flow is performed here. The potential value between the reference electrode 6 and the counter electrode 5 is determined, i.e. the relative potential difference between the two electrodes 2 is measured.

The measured OCP quantity and/or a value derived therefrom can be output as a diagnostic value.

An amperometric measurement is performed in the next step of the regeneration method. To do this, the connection of the electrodes 2 is modified. As shown in FIG. 5, the reference electrode 6 is energized. The potential to be applied for this purpose is derived from the addition of the measured values of the previous steps, i.e. from the addition of the peak of the linear sweep measurement and the potential between the counter electrode 5 and the reference electrode 6.

The measured quantity, for example a time period up to the end of the amperometric measurement, and/or a value derived therefrom can be output as a diagnostic value.

In one embodiment (not shown), a self-test is carried out to monitor the regeneration method by means of the linear sweep method in order to check the effectiveness of the method and/or to determine the result of the regeneration.

The regeneration method is initiated until the current flow indicates a complete substance conversion. Ideally, the potential value of the reference electrode 5 should be −300±50 mV compared with the potential value before regeneration, so that the equilibrium position is restored.

Each of the connection diagrams described above represents a connection of the at least two electrodes 2. The connection device 8 is configured, in a manner not shown in detail, by means of switchover devices and other switches, to switch between the connections in response to single control signals or a plurality of control signals. The described processes or further processes can thus be implemented automatically by achieving conditions such as the stationarity of measured values, by reaching threshold values of the measured values and/or through expiration of time intervals.

The invention thus relates to a regeneration method for an electrochemical sensor 1, wherein the sensor 1 comprises at least two electrodes 2, wherein the at least two electrodes 2 are operated in a first connection in a normal operation of the sensor 1. The regeneration method differs from normal operation in terms of a modified connection of the at least two electrodes 2.

### REFERENCE SIGN LIST

1 Sensor
2 Electrode
3 Sensing electrode
4 Auxiliary electrode
5 Counter electrode
6 Reference electrode
7 Voltage source
8 Connection device
9 Sensor arrangement

1. A regeneration method for an electrochemical sensor (1), wherein the sensor (1) comprises at least two electrodes (2) that are operable in a first connection in a normal operation of the sensor (1), the regeneration method comprising:
modifying a connection of the at least two electrodes (2) from the normal operation to form a modified connection of the at least two electrodes (2), and
effecting at least one substance conversion at the at least two electrodes (2) with the modified connection which differs from the normal operation.

2. The regeneration method as claimed in claim 1, wherein at least one first electrode of the at least two electrodes (2) is suitable for detecting contamination of a second electrode of the at least two electrodes (2).

3. The regeneration method as claimed in claim 1, further comprising using a first electrode (2) of the at least two electrodes (2) for a voltage measurement in the normal operation, and the modifying of the connection comprises applying a current to the first electrode (2), and the generating of the substance conversion occurs at the first electrode (2).

4. The regeneration method as claimed in claim 3, wherein the substance conversion does not take place at the first electrode (2) in the normal operation.

5. The regeneration method as claimed in claim 1, further comprising determining an amount of an applied current by a self-test of the sensor (1).

6. The regeneration method as claimed in claim 1, further comprising determining a current flow as a function of an applied voltage.

7. The regeneration method as claimed in claim 1, wherein the at least two electrodes (2) comprises at least three electrodes (2), and the method further comprising determining a current flow as a function of an applied voltage between two of the at least three electrodes (2) of the sensor (1).

8. The regeneration method as claimed in claim 7, wherein the current flow is determined as a function of the applied voltage between a counter electrode (5) of the at least three electrodes and an auxiliary electrode (4) of the at least three electrodes or between a sensing electrode (3) of the at least three electrodes and the auxiliary electrode (4).

9. The regeneration method as claimed in claim 7, further comprising measuring a relative potential difference between two of the at least three electrodes (2) of the sensor (1).

10. The regeneration method as claimed in claim 9, wherein the relative potential difference between a first electrode (2) of the at least three electrodes (2) and a second or third electrode (2) of the at least three electrodes (2) of the sensor is measured.

11. The regeneration method as claimed in claim 9, wherein the relative potential difference between a reference electrode (6) of the at least three electrodes (2) and a counter electrode (5) of the at least three electrodes (2) of the sensor (1) is measured.

12. The regeneration method as claimed in claim 1, further comprising, in at least in the modified connection, energizing at least one of the electrodes (2) amperometrically.

13. The regeneration method as claimed in claim 1, further comprising determining a current flow as a function of an applied voltage, and then determining an amount of an applied current based on the determined current flow.

14. The regeneration method as claimed in claim 13, wherein the determining of the amount of the applied current based on the determined current flow is based on a determined voltage value for an extreme value of the current flow and/or a determined relative potential difference.

15. The regeneration method as claimed in claim 1, further comprising carrying out a self-test to monitor the regeneration method.

16. The regeneration method as claimed in claim 1, wherein the at least two electrodes (2) include a first electrode (2) which is a sensing electrode (3), a second electrode (2) which is an auxiliary electrode (4), a third electrode (2) which is a counter electrode (5) and/or a fourth electrode (2) which is a reference electrode (6) of the sensor (1).

17. The regeneration method as claimed in claim 1, wherein the sensor (1) is at least one of an amperometric gas sensor, a nitrogen monoxide sensor, a carbon monoxide sensor, or an oxygen sensor.

18. A sensor arrangement (8) having at least one electrochemical sensor (1), the at least one sensor (1) comprises:
at least two electrodes (2);
a connection device (7) for changing between at least first and second connections of the at least two electrodes (2), wherein the first connection of the at least two connections is configured for a normal operation and the second connection of the at least two electrodes is a modified connection configured for carrying out a regeneration method.

19. The sensor arrangement (8) as claimed in claim 18, wherein the connection device (7) is configured for an automatic changeover between the at least first and second connections, in particular between at least three or at least four connections.

20. The sensor arrangement (8) as claimed in claim 19, wherein the automatic changeover is a condition-controlled automatic changeover.
